(19) United States
(12) Patent Application Publication
(10) Pub. No.: US 2023/0132048 A1
(43) Pub. Date: Apr. 27, 2023

(54) METHOD FOR PROVIDING INFORMATION ABOUT THERAPEUTIC RESPONSE TO ANTICANCER IMMUNOTHERAPY, AND KIT USING SAME

(71) Applicant: INDUSTRY-ACADEMIC COOPERATION FOUNDATION, YONSEI UNIVERSITY, Seoul (KR)

(72) Inventors: Hye Ryun KIM, Seoul (KR); In Suk LEE, Seoul (KR); Jae Won CHO, Seoul (KR)

(73) Assignee: INDUSTRY-ACADEMIC COOPERATION FOUNDATION, YONSEI UNIVERSITY, Seoul (KR)

(21) Appl. No.: 17/611,988

(22) PCT Filed: Apr. 28, 2020
(86) PCT No.: PCT/KR2020/005625

§ 371 Date: Nov. 17, 2021

(30) Foreign Application Priority Data
May 17, 2019 (KR) 10-2019-0057913

(51) Int. Cl. C12Q 1/6886

(52) U.S. Cl. CPC C12Q 1/6886; C12Q 2600/154; C12Q 2600/106

(57) ABSTRACT

The present specification provides a method for providing information about a therapeutic response to anticancer immunotherapy, and a kit for providing information by using same, the method comprising: measuring DNA methylation of pDMRs in a biological sample isolated from a subject; and evaluating the therapeutic reaction to anticancer immunotherapy for the subject on the basis of the measured DNA methylation of pDMRs.

### CROSS-REFERENCE TO RELATED APPLICATIONS

This application is a 371 National Stage filing and claims the benefit under 35 U.S.C. § 120 to International Application No. PCT/KR2020/005625, filed 28 Apr. 2020, which claims priority to Korean Application No. 10-2019-0057913, filed 17 May 2019, each of which is incorporated herein by reference in its entirety.

### SEQUENCE LISTING

This application herein incorporates by reference in its entirety the Sequence Listing material in the ASCII text file named “19PD5036PCT-US_Sequence Listing_ST25”, created Jun. 16, 2022 and having the size of 1235 bytes.

### TECHNICAL FIELD

The present invention relates to a method for providing information about a therapeutic response to anticancer immunotherapy, and more specifically, to a method for providing information about a therapeutic response to anticancer immunotherapy for non-small lung cancer using biomarkers and a kit based on the same.

### BACKGROUND ART

Lung cancer is one of the most common cancers in both sexes. Among lung cancers, non-small lung cancer (NSLC) is a type of epithelial carcinoma and refers to all epithelial lung cancers other than small lung cancer. The non-small lung cancer accounts for a high percentage of a total incidence of lung cancers.

In this connection, the non-small lung cancer is classified into several subtypes according to the size, shape, and chemical composition of tumor cells. Representative examples thereof include adenocarcinoma, squamous cell carcinoma, large cell carcinoma, and the like. Adenocarcinoma is found in the outer region of the lung and tends to progress more slowly than other lung cancers but has a high tendency to metastasize at an early stage and also has high radiation resistance. Squamous cell carcinoma begins in the early version of the cells forming the airway and has a high incidence mainly in smokers. Furthermore, large cell carcinoma can develop anywhere in the lung, and progression thereof is fast enough to be similar to that of small cell lung cancer, and the treatment thereof has emerged as a challenge to date.

Symptoms of such non-small lung cancer include persistent cough, chest pain, weight loss, nail damage, joint pain, shortness of breath, and the like. However, since non-small lung cancer progresses more slowly than other cancers, it rarely exhibits symptoms at the beginning stage thereof. Therefore, early detection and treatment of non-small lung cancer are difficult. NSLC is highly likely to be detected after metastasis to the whole body, such as bone, liver, small intestine, and brain. Therefore, when diagnosing the non-small lung cancer, greater than half of the patients are in a metastasis state enough to be unable to perform surgery. Thus, early treatment thereof is practically difficult. Further, when non-small cell cancer is not in the metastasis state enough to perform surgical operation, prior surgery such as radical resection is performed. However, only about 30% of cases can be subject to radical resection. Furthermore, it is shown that in the majority of all patients who underwent radical resection, more aggressive cancer can recur after the surgical resection, resulting in death.

For this reason, for the early treatment of non-small lung cancer, the development of a new treatment method and further development of a new method to predict the therapeutic response to the existing treatment method are continuously required.

This Background Art section is written to facilitate understanding of the present invention. It should not be appreciated that the matters described in Background Art section are regarded as prior art.

### DETAILED DESCRIPTION OF THE INVENTION

### Technical Problems

The use of an immune checkpoint blockade has been proposed as a treatment method for non-small lung cancer. In particular, PD-1 (programmed cell death-1)/PD-L1 (programmed cell death ligand-1) blockage as approved by the Food and Drug Administration was shown to be effective in the treatment of non-small lung cancer.

In predicting the therapeutic response to PD-L1 blockage, tumor PD-L1 expression by immunohistochemistry (IHC) can be used as the best prediction biomarker for PD-1 blockage. However, the accuracy of prediction of the therapeutic response to PD-L1 which is dependent on PD-L1 expression in tumor cells is not high enough to confirm drug efficacy. More specifically, patients with PD-L1 expression negative can respond to PD-1 blockage, patients with PD-L1 expression positive cannot respond to PD-1 blockage. Furthermore, some responding patients without PD-L1 can have a response duration similar to that of PD-L1 positive in clinical trials (e.g., Checkmate 057). Moreover, PD-L1 expression can be dynamic, and can change temporally and spatially. This change in PD-L1 expression can be adaptive immune resistant as exerted by tumor.

The inventors of the present invention noted that epigenetic regulation of tumors through DNA methylation is associated not only with immunogenicity recovery but also with immune evasion of cancer cells, and methylation-based biomarkers have many advantages over genomic and transcriptional biomarkers, including stability and resistance to heterogeneity in tumor samples.

Accordingly, in order to select candidates with strong predictive power, the present inventors integrated DNA methylation and mRNA expression data and selected differentially methylated regions (DMRs) that were more functionally related based on strict criteria. As a result, promoter associated differentially methylated regions (pDMRs) for CYTIP and TNFSF8 genes were found as candidate biomarkers for subsequent validation.

In particular, the inventors of the present invention noted that DNA methylation of pDMRs for CYTIP and TNFSF8 genes showed significant differences depending on responders and non-responders to anti-PD-1 therapy, an anticancer immunotherapy for non-small lung cancer. As a result, the inventors of the present invention have come to develop a new method for providing information capable of predicting responders or non-responders to anti-PD-1 therapy by measuring DNA methylation of pDMRs for CYTIP and TNFSF8. In particular, this method can be utilized as a biomarker that can be used independently other than PD-L1 as it has been shown to have better predictive ability than tumor PD-L1 expression, which is a conventional method for predicting the response to anti-PD-1 therapy.

Accordingly, a technical problem of the present invention is to provide a method for providing information about a therapeutic response to anticancer immunotherapy, in which the method is configured to measure DNA methylation of pDMRs for CYTIP and TNFSF8 genes in a biological sample isolated from a subject, and predict the therapeutic response to anticancer immunotherapy, particularly anti-PD-1 therapy, based on the DNA methylation of their pDMRs, and a kit using the same.

Another technical problem of the present invention is to provide a method for providing information about a therapeutic response to anticancer immunotherapy, in which the method is configured to measure DNA methylation of pDMRs for CYTIP and TNFSF8 genes in a biological sample isolated from a subject before anticancer immunotherapy is performed, and predict the therapeutic response to anticancer immunotherapy more accurately based on a predetermined threshold for DNA methylation of pDMRs, and a kit using the same.

Another technical problem of the present invention is to provide a method for providing information about a therapeutic response to anticancer immunotherapy, in which the method can present anticancer immunotherapy that is predicted to have good prognosis for each subject, based on the DNA methylation of pDMRs for CYTIP and TNFSF8 genes measured from a biological sample, and a kit using the same.

Another technical problem of the present invention is to provide a method for providing information about a therapeutic response to anticancer immunotherapy, in which the method can provide information related to diagnosis for lung cancer, especially non-small lung cancer, and a kit using the same.

The problems of the present invention are not limited to the problems mentioned above. Other problems not mentioned will be clearly understood by those skilled in the art from the following description.

### Means for Solving the Problems

According to an embodiment of the present invention, there is provided a method for providing information about a therapeutic response to anticancer immunotherapy, in which the method includes: measuring DNA methylation of pDMRs in a biological sample isolated from a subject, preferably measuring DNA methylation of pDMRs of at least one gene from the group consisting of CYTIP and TNFSF8; and evaluating the therapeutic response to anticancer immunotherapy for the subject on the basis of the DNA methylation of pDMRs.

The measuring DNA methylation of pDMRs can include the measuring the presence or absence of DNA methylation or level of pDMRs.

According to one feature of the present invention, the subject is a subject suspected of non-small lung cancer. The biological sample can include at least one selected from the group consisting of tumor tissue, peripheral blood, serum, and plasma.

As used herein, the term “non-small lung cancer” is a type of epithelial cancer and refers to all epithelial lung cancers other than small lung cancer. In one example, anticancer immunotherapy for such non-small lung cancer can include anti-PD-1 therapy, but is not limited thereto, and can include at least one selected from the group consisting of anti-CTLA-4 therapy, anti-CD28 therapy, anti-KIR therapy, anti-TCR therapy, anti-LAG-3 therapy, anti-TIM-3 therapy, anti-TIGIT therapy, anti-A2aR therapy, anti-ICOS therapy, anti-OX40 therapy, anti-4-1BB therapy, and anti-GITR therapy.

As used herein, the term “anti-PD-1 therapy” can be a treatment method configured to block a mechanism by which T cells cannot attack tumor cells. More specifically, anti-PD-1 therapy can be based on preventing the binding of PD-L1 and PD-L2 as immune checkpoint ligands of surface proteins of the tumor cell and with PD-1 as an immune checkpoint receptor of proteins on the surface of T cells. For example, when an immuno-anticancer drug binds to the PD-1 receptor of T cells, it can inhibit the evasion function of T cells from the tumor cells, and as a result, the activated T cells can kill the tumor cells. Therefore, in the present invention, “anti-PD-1 therapy” can be used with the same meaning as “PD-1 pathway blockage.”

As used herein, the term “pDMRs (promoter associated differentially methylated regions)” refers to regions in which the presence or absence of DNA methylation of a promoter for a gene differs between a responder and a non-responder tumor tissue to anticancer immunotherapy, or regions with different levels of methylation. However, the pDMRs are not limited thereto, and can include DNA regions having different methylation states among tissues, cells, and subjects. In addition, DMR can be a functional region capable of regulating gene transcription, and identification of DMR may provide information on epigenetic differences in human tissues.

DNA methylation of pDMRs for CYTIP and TNFSF8 genes in a biological sample obtained from a subject can be used as a biomarker for predicting response to anticancer immunotherapy, particularly, anti-PD-1 therapy. In this connection, the biological sample can be a tumor tissue obtained from a subject before anticancer immunotherapy is performed but is not limited thereto. Furthermore, the aforementioned biomarker can be used independently of each other or in various combinations for predicting a therapeutic response.

In various embodiments of the present invention, DNA methylation of pDMRs for CYTIP and TNFSF8 genes in a tumor tissue obtained from a subject before anticancer immunotherapy is performed can be used for determining whether a subject responds positively or negatively to treatment with anticancer immunotherapy.

According to an embodiment of the present invention, when the DNA methylation level of pDMRs for a CYTIP gene in a subject before anticancer immunotherapy is performed is less than a predetermined level, the subject can be evaluated as a positive for a therapeutic response to anticancer immunotherapy, and when the level is greater than or equal to a predetermined level, the subject can be evaluated as a negative for a therapeutic response to anticancer immunotherapy. In this connection, the DNA methylation level of pDMRs for the CYTIP gene predetermined in the evaluation of a therapeutic response can be 50%. However, it is not limited thereto.

According to another embodiment of the present invention, when the DNA methylation level of pDMRs for a TNFSF8 gene in a subject before anticancer immunotherapy is performed is less than a predetermined level, the subject can be evaluated as a positive for a therapeutic response to anticancer immunotherapy, and when the level is greater than or equal to a predetermined level, the subject can be evaluated as a negative for a therapeutic response to anticancer immunotherapy. In this connection, the DNA methylation level of pDMRs for the TNFSF8 gene predetermined in the evaluation of a therapeutic response can be 60%. However, it is not limited thereto.

According to another embodiment of the present invention, when the DNA methylation level of pDMRs for a combination of CYTIP and TNFSF8 genes in a subject before anticancer immunotherapy is performed is less than a predetermined level, the subject can be evaluated as a positive for a therapeutic response to anticancer immunotherapy, and when the level is greater than or equal to a predetermined level, the subject can be evaluated as a negative for a therapeutic response to anticancer immunotherapy.

As used herein, the term “positive therapeutic response” can refer to the occurrence of a response in which a receptor on the surface of T cells is prevented from binding to a ligand on the surface of a tumor cell by immune checkpoint blockades such as PD-1 blockade. However, the disclosure is not limited thereto. The positive therapeutic response can include the occurrence of any response associated with a favorable prognosis or alleviation of symptoms of non-small lung cancer by an immune checkpoint blockade. Therefore, for subjects with positive therapeutic response to anticancer immunotherapy, symptoms of non-small lung cancer can be alleviated by anticancer immunotherapy. Subjects with negative therapeutic response to anticancer immunotherapy can have relatively poor prognosis following the anticancer immunotherapy.

According to another embodiment of the present invention, there is provided a kit for providing information about a therapeutic response to anticancer immunotherapy, in which the kit includes formulations configured to measure DNA methylation of pDMRs in a biological sample isolated from a subject, preferably formulations configured to measure DNA methylation of pDMRs of at least one gene from the group consisting of CYTIP and TNFSF8.

According to one feature of the present invention, the kit for providing information is configured to present positive or negative therapeutic response to anticancer immunotherapy based on DNA methylation of pDMRs of at least one gene from the group consisting of CYTIP and TNFSF8, preferably the presence or absence of DNA methylation or level of pDMRs. The anticancer immunotherapy can be anti-PD-1 therapy but is not limited thereto.

The subject is a subject suspected of non-small lung cancer. The biological sample can include at least one selected from the group consisting of tumor tissue, blood, serum, and plasma and can be isolated from the subject before the anticancer immunotherapy is performed but is not limited thereto.

Furthermore, the kit according to another embodiment of the present invention can be configured to provide further information on increasing clinical benefit, such as overall survival (OS), progression-free survival (PFS), complete response (CR), or partial response (PR).

According to one feature of the present invention, when the DNA methylation level of pDMRs for a CYTIP gene in a biological sample obtained from a subject before anticancer immunotherapy is performed is less than 50%, the kit can be configured to present a positive therapeutic response to anticancer immunotherapy, and when the DNA methylation level of pDMRs for a CYTIP gene is greater than or equal to 50%, the kit can be configured to present a negative therapeutic response to anticancer immunotherapy.

According to another feature of the present invention, when the DNA methylation level of pDMRs for a TNFSF8 gene in a biological sample obtained from a subject before anticancer immunotherapy is performed is less than 60%, the kit can be configured to present a positive therapeutic response to anticancer immunotherapy, and when the DNA methylation level of pDMRs for a TNFSF8 gene is greater than or equal to 60%, the kit can be configured to present a negative therapeutic response to anticancer immunotherapy.

Hereinafter, the present invention will be described in more detail based on Examples. However, since these Examples are merely for illustrative purposes of the present invention, the scope of the present invention should not be construed as being limited to these Examples.

### Effects of the Invention

The present invention has the effect of providing a new biomarker capable of predicting a therapeutic response to anticancer immunotherapy, especially PD-1 blockage.

More specifically, the present invention can provide a biomarker with high accuracy in predicting treatment responsiveness with respect to a biological sample obtained from a subject before anticancer immunotherapy, preferably anti-PD-1 therapy, is performed. Accordingly, the present invention can provide information that can be effective for accurate diagnosis, rather than a conventional prediction method based on biomarkers for predicting the responsiveness of an anti-PD-1 therapy.

Furthermore, the present invention has the effect of providing anticancer immunotherapy that can be more effective, based on a DNA methylation measurement for a biomarker. For example, the present invention can distinguish subjects having positive therapeutic responses to anti-PD-1 from subjects having negative therapeutic responses to anti-PD-1, and an effective treatment can be selected depending on whether the subject has a positive or negative therapeutic response.

The effect of the present invention is not limited by the contents illustrated above. Further effects are included within the present specification.

### BRIEF DESCRIPTION OF THE DRAWINGS

FIG. 1 shows an exemplary procedure of a method for providing information about a therapeutic response to anticancer immunotherapy according to an embodiment of the present invention.

FIGS. 2A and 2B show the evaluation results for responders and non-responders to anti-PD-1 therapy, which is anticancer immunotherapy, according to the DNA methylation level of pDMRs for CYTIP and TNFSF8 genes and PD-L1 expression pattern in a tumor tissue of non-small lung cancer patients.

FIGS. 3A and 3B show the evaluation results for response prediction to anti-PD-1 therapy, which is anticancer immunotherapy, for non-small lung cancer patients, according to the DNA methylation level of pDMRs for CYTIP and TNFSF8 genes and PD-L1 expression pattern.

FIGS. 4A to 4E show the steps of determining the pDMRs of a gene used as a biomarker of the present invention in a tumor tissue of non-small lung cancer patients.

### BEST MODES FOR CARRYING OUT THE INVENTION

Advantages and features of the present invention and a method of achieving them will become apparent with reference to the embodiments described later in detail together with the accompanying drawings. However, the present invention is not limited to the embodiments disclosed below but will be implemented in various different forms. Only these embodiments make the disclosure of the present invention complete and are provided to completely inform the scope of the disclosure to those with ordinary knowledge in the technical field to which the present invention belongs. The present invention is only defined by the scope of the claims.

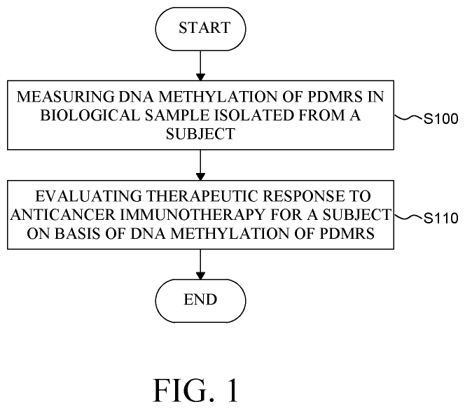

Hereinafter, a procedure of a method for providing information about a therapeutic response to anticancer immunotherapy according to an embodiment of the present invention will be described in detail with reference to FIG. 1.

FIG. 1 shows an exemplary procedure of a method for providing information about a therapeutic response to anticancer immunotherapy according to an embodiment of the present invention.

Referring to FIG. 1, a method for providing information about a therapeutic response to anticancer immunotherapy according to an embodiment of the present invention is configured first to measure DNA methylation of pDMRs in a biological sample isolated from a subject (S100) and evaluate a therapeutic response to anticancer immunotherapy for the subject, based on the level of the measured DNA methylation of pDMRs (S110).

According to an embodiment of the present invention, the step of measuring the DNA methylation of pDMRs (S100) can include measuring DNA methylation of pDMRs for at least one gene from the group consisting of CYTIP and TNFSF8 in a biological sample of tumor tissue, blood, serum, or plasma isolated from a subject suspected of the non-small lung cancer.

According to an embodiment of the present invention, the step of evaluating the therapeutic response to anticancer immunotherapy (S110) can include: when the measured DNA methylation level of pDMRs for at least one gene in the group consisting of CYTIP and TNFSF8 is less than a predetermined level, determining a subject as positive; when the measured DNA methylation level is greater than or equal to a predetermined level, determining the subject as negative; and evaluating the therapeutic response to the anticancer immunotherapy, based on whether the DNA methylation of pDMRs is positive or negative.

In this connection, the anticancer immunotherapy can be anti-PD-1 therapy but is not limited thereto. For example, the anticancer immunotherapy can be at least one therapy selected from the group consisting of anti-CTLA-4 therapy, anti-CD28 therapy, anti-KIR therapy, anti-TCR therapy, anti-LAG-3 therapy, anti-TIM-3 therapy, anti-TIGIT therapy, anti-A2aR therapy, anti-ICOS therapy, anti-OX40 therapy, anti-4-1BB therapy, and anti-GITR therapy.

According to another embodiment of the present invention, the step of evaluating the therapeutic response to anticancer immunotherapy (S110) can include: when the DNA methylation level of pDMRs for a CYTIP gene in a biological sample is less than 50%, evaluating a subject as positive for a therapeutic response to the anticancer immunotherapy.

According to another embodiment of the present invention, the step of evaluating the therapeutic response to anticancer immunotherapy (S110) can include: when the DNA methylation level of pDMRs for a TNFSF8 gene in a biological sample is less than 60%, evaluating a subject as positive for a therapeutic response to the anticancer immunotherapy.

According to another embodiment of the present invention, the step of evaluating the therapeutic response to anticancer immunotherapy (S110) can include: when the DNA methylation level of pDMRs for a combination of CYTIP and TNFSF8 genes in a biological sample is less than a predetermined level, evaluating the subject as positive for a therapeutic response to the anticancer immunotherapy.

According to the above procedure, the method for providing information about a therapeutic response according to an embodiment of the present invention can measure the levels of various markers, and thus can provide information to allow a therapeutic response to the anticancer immunotherapy for a subject, particularly, a therapeutic response to anti-PD-1 to be early predicted.

### Example 1: Prediction of Therapeutic Response to PD-1 Blockade Based on DNA Methylation of pDMRs

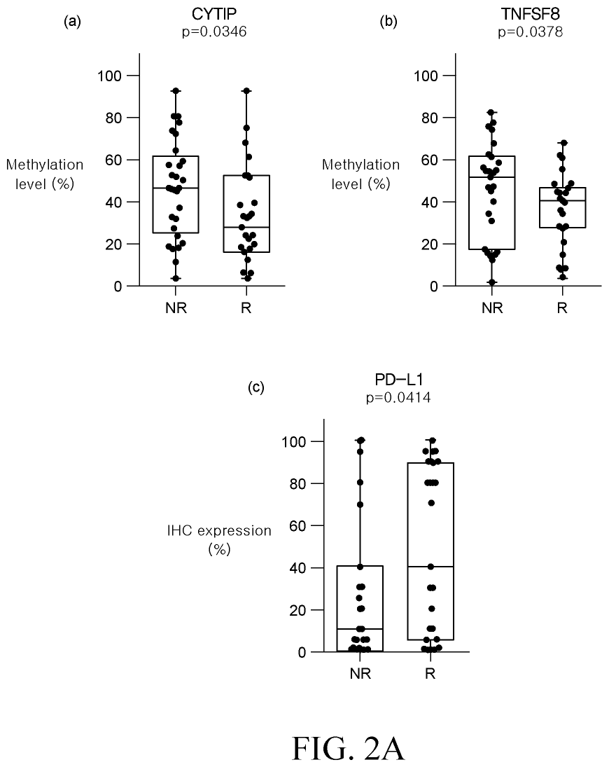

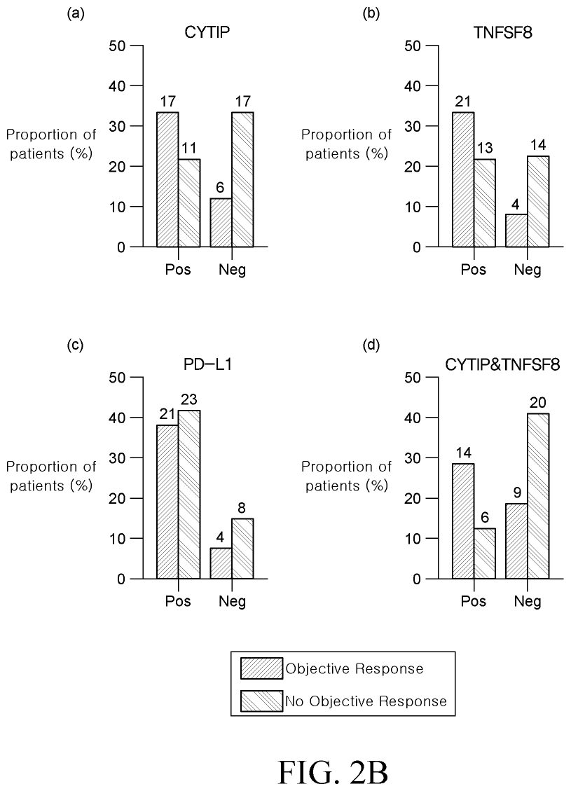

Hereinafter, with reference to FIGS. 2A and 2B, biomarkers used for prediction method of therapeutic responses to the anticancer immunotherapy, in particular, anti-PD-1 therapy, according to various embodiments of the present invention, and therapeutic response prediction method using the same will be described.

FIGS. 2A and 2B show the evaluation results of predicting a therapy response to anti-PD-1, which is anticancer immunotherapy, for non-small lung cancer patients, according to the level of the biomarker used in various embodiments of the present invention.

Referring to (a) and (b) of FIGS. 2A, the DNA methylation level of pDMRs for CYTIP and TNFSF8 genes in the tumor tissues obtained from non-small lung cancer patients before anti-PD-1 therapy was performed was divided into responders (R) and non-responders (NR) to anti-PD-1 therapy and presented with box and whisker data. In addition, referring to (c) of FIG. 2A, the PD-L1 expression rate in the tumor tissues obtained from non-small lung cancer patients before the anti-PD-1 therapy was performed was divided into responders (R) and non-responders (NR) to anti-PD-1 therapy and presented with box and whisker data. The end of each box represents the proportions of 25% and 75% of the group, the midline represents the proportion of 50%, and the whiskers at the end of the box represent the highest and lowest values of each group.

Furthermore, the positive or negative discrimination level of the response to anti-PD-1 therapy through the DNA methylation level of pDMR is determined as a threshold level at which each ratio is maximized when both a positive predictive value (PPV; #true responders) and a negative predictive value (NPV; #true non-responders) are taken into account. In this connection, the positive predictive value is defined as a ratio of responders below the threshold level among responders and non-responders to the anti-PD-1 therapy, and the negative predictive value is defined as a ratio of non-responders above the threshold level among responders and non-responders to the anti-PD-1 therapy.

Referring to (a) of FIG. 2A, the DNA methylation level of pDMRs for the CYTIP gene in the responders and the non-responders showed a significant (p=0.0346) difference. In addition, the distribution of DNA methylation level values of pDMRs for the CYTIP gene in the non-responders showed an overall high methylation level, and the distribution of the DNA methylation level values of pDMRs for the CYTIP gene in the responders showed an overall low methylation level. In this connection, the threshold level at which the ratio of the positive predictive value to the negative predictive value is maximized can be 40%. In various embodiments, the threshold level determining whether a response to anti-PD-1 therapy is positive or negative can be 40%, but is not limited thereto, and may preferably be 20%, 30%, 50%, or 60%. Based thereon, when the DNA methylation level of pDMRs for the CYTIP gene is less than the threshold level, the response to the anti-PD-1 therapy, which is an anticancer immunotherapy, can be evaluated as positive, and when the DNA methylation level of pDMRs for the CYTIP gene is greater than or equal to the threshold level, the response to the anti-PD-1 therapy can be evaluated as negative.

Referring to (b) of FIG. 2A, the DNA methylation level of pDMRs for the TNFSF8 gene in the responders and the non-responders showed a significant (p=0.0378) difference. In addition, the distribution of DNA methylation level values of pDMRs for the TNFSF8 gene in the non-responders showed an overall high methylation level, and the distribution of the DNA methylation level values of pDMRs for the TNFSF8 gene in the responders showed an overall low methylation level. In this connection, the threshold level at which the ratio of the positive predictive value to the negative predictive value is maximized can be 50%. In various embodiments, the threshold level determining whether a response to anti-PD-1 therapy is positive or negative can be 50%, but is not limited thereto, and may preferably be 30%, 40%, 60%, or 70%. Based thereon, when the DNA methylation level of pDMRs for the TNFSF8 gene is less than the threshold level, the response to the anti-PD-1 therapy, which is an anticancer immunotherapy, can be evaluated as positive, and when the DNA methylation level of pDMRs for the TNFSF8 gene is equal to or the threshold level, the response to the anti-PD-1 therapy can be evaluated as negative.

Referring to (c) of FIG. 2A, the PD-L1 expression rate between the responders and the non-responders is shown as some significant (p=0.0414) difference. However, in comparison with the distribution of the DNA methylation level values of pDMRs for the CYTIP and TNFSF8 genes in the responders and non-responders described above in (a) and (b) of FIG. 2A, it is shown that in the distribution of the PD-L1 expression rate in the non-responders, the majority of them have low methylation level values, and the responders have a sporadic distribution. Accordingly, these results show a significant difference relatively lower than the DNA methylation results of pDMRs for CYTIP and TNFSF8 genes in the responders and non-responders described above in (a) and (b) of FIG. 2A. In other words, it is shown that DNA methylation of pDMRs for CYTIP and TNFSF8 genes has a higher correlation in predicting response to anti-PD-1 therapy than PD-L1 expression, which is conventional anti-PD-1 therapy response prediction.

Referring to (a), (b), (c) and (d) of FIG. 2B, the positive predictive value and negative predictive value of anti-PD-1 therapy, which is anticancer immunotherapy, according to the DNA methylation level of pDMRs for CYTIP and TNFSF genes and PD-L1 expression are shown.

Referring to (a) of FIG. 2B, the positive predictive value determined through the DNA methylation level of pDMRs for the CYTIP gene was 60.7% ( 17/28), and the negative predictive value was 73.9% ( 17/23). Furthermore, referring to (b) of FIG. 2B, the positive predictive value determined through the DNA methylation level of pDMRs for the TNFSF8 gene was 61.8% ( 21/34), and the negative predictive value was 77.8% ( 14/18). Furthermore, the positive predictive value determined through the DNA methylation level of pDMRs for a combination of the two genes was 70% ( 14/20), and the negative predictive value was 69% ( 20/29). On the other hand, the positive predictive value determined through the PD-L1 expression rate was 47.7% ( 21/44), and the negative predictive value was 66.7% ( 8/12), which is shown to be relatively lower than the positive predictive value and the negative predictive value determined through the aforementioned DNA methylation.

Based on a result of Example 1 above, the DNA methylation of pDMRs can be used as biomarkers having a better predictive power than PD-L1, which is a conventional method for prediction of an anti-PD-1 therapeutic response in a method for providing information about a therapeutic response according to various embodiments of the present invention.

### Example 2: Prediction of Therapeutic Response to PD-1 Blockade Based on DNA Methylation of pDMRs for Non-Small Lung Cancer Patients

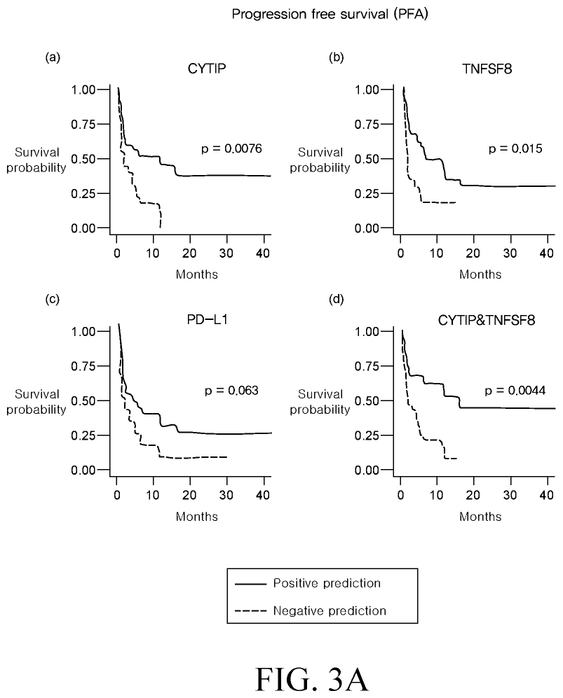

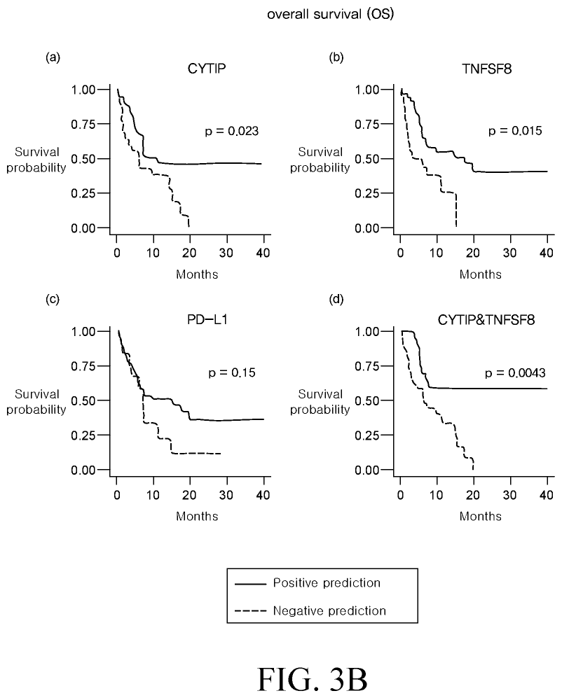

Hereinafter, with reference to FIGS. 3A and 3B, based on the DNA methylation level and PD-L1 expression of pDMRs for CYTIP and TNFSF8 in tumor tissues obtained from non-small lung cancer patients, the evaluation results for predicting anticancer immunotherapy response will be described. In this connection, the biological sample used is a tumor tissue obtained from a non-small lung cancer patient but is not limited thereto.

FIGS. 3A and 3B show the evaluation results for response prediction to anti-PD-1 therapy, which is anticancer immunotherapy, of non-small lung cancer patients, according to the DNA methylation level and PD-L1 expression pattern of pDMRs for CYTIP and TNFSF8 genes.

Referring to (a) of FIG. 3A, the progression free survival (PFS) of the positive predictive group and the negative predictive group according to the DNA methylation level of pDMRs for the CYTIP gene is shown. There was a significant (p=0.076) difference between the positive predictive group and the negative predictive group, and the positive predictive group showed significantly longer progression free survival than the negative predictive group.

Furthermore, referring to (b) of FIG. 3A, the positive predictive group and the negative predictive group according to the DNA methylation level of pDMRs for the TNFSF8 gene showed a significant (p=0.015) difference, and the positive predictive group showed significantly longer progression free survival than the negative predictive group.

Furthermore, referring to (d) of FIG. 3A, the positive predictive group and the negative predictive group according to the DNA methylation level of pDMRs for a combination of CYTIP and TNFSF8 genes showed a significant (p=0.0044) difference, and the positive predictive group showed significantly longer progression free survival than the negative predictive group.

In comparison, referring to (c) of FIG. 3A, there was no significant (p=0.063) difference between the positive predictive group and the negative predictive group according to PD-L1 expression.

Referring to (a) of FIG. 3B, overall survival (OS) of the positive predictive group and the negative predictive group according to the DNA methylation level of pDMRs for the CYTIP gene is shown. There was a significant (p=0.023) difference between the positive predictive group and the negative predictive group, and the positive predictive group showed significantly longer overall survival than the negative predictive group.

Furthermore, referring to (b) of FIG. 3B, the positive predictive group and the negative predictive group according to the DNA methylation level of pDMRs for the TNFSF8 gene showed a significant (p=0.015) difference, and the positive predictive group showed significantly longer overall survival than the negative predictive group.

Furthermore, referring to (d) of FIG. 3B, the positive predictive group and the negative predictive group according to the DNA methylation level of pDMRs for a combination of CYTIP and TNFSF8 genes showed a significant (p=0.0043) difference, and the positive predictive group showed significantly longer overall survival than the negative predictive group.

In comparison, referring to (c) of FIG. 3B, there was no significant (p=0.15) difference between the positive predictive group and the negative predictive group according to PD-L1 expression.

Based on a result of Example 2 above, this result can mean that DNA methylation of pDMRs can be a better marker than conventional PD-L1 expression in predicting response to anti-PD-1 therapy, which is anticancer immunotherapy, in non-small lung cancer patients.

### Example 3: Setting Up Biomarkers for Predicting Response to Anti-PD-1 Therapy, which is Anticancer Immunotherapy

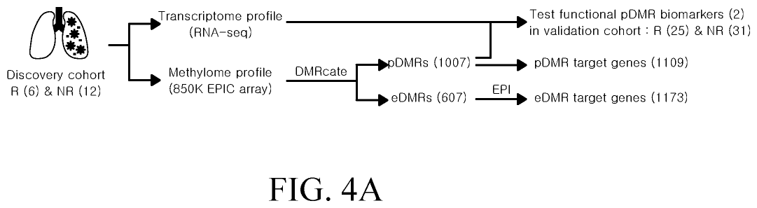

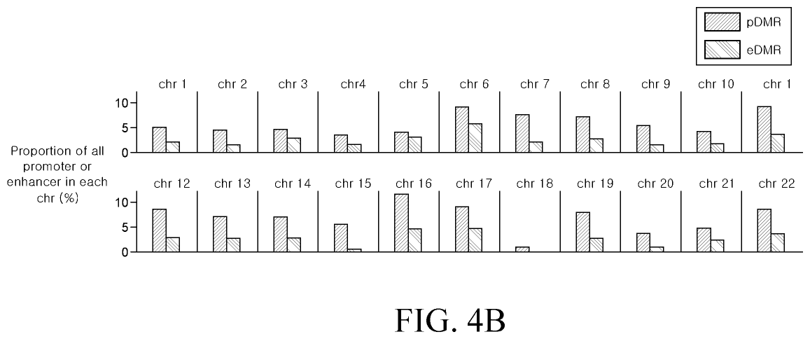

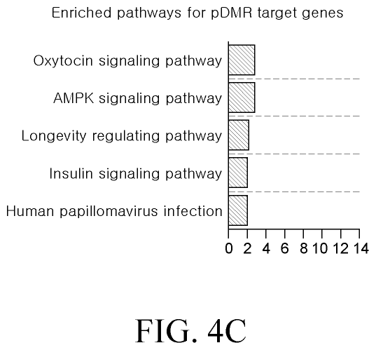

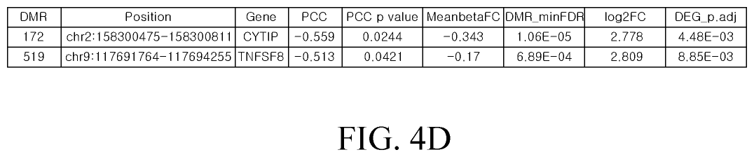

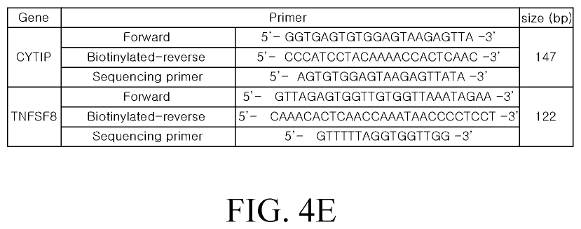

Hereinafter, with reference to FIG. 4, methods for classifying methylation of genes related to response to anti-PD-1 therapy, which is anticancer immunotherapy, in tumor tissues of non-small lung cancer patients, and setting up biomarkers for predicting a therapeutic response of anticancer immunotherapy using the same will be described.

FIGS. 4A to 4E show the classification process of setting up pDMRs for CYTIP and TNFSF8 genes used as biomarkers in various embodiments of the present invention.

Referring to FIG. 4A, for the classification for setting up pDMRs, a transcriptome profile and a methylome profile were measured for 6 responders and 12 non-responders to anti-PD-1 therapy, which is anticancer immunotherapy, and DMRs were identified through DMRcate and classified into 1007 pDMRs and 607 eDMRs related to response to anti-PD-1 therapy. Furthermore, 1109 pDMR target genes and 1176 eDMR target genes were selected in consideration of promoter and enhancer-promoter interaction (EPI) in the consensus coding gene sequence (CCDS) database.

These results are shown as the ratio of pDMR and eDMR associated with the response to anti-PD-1 therapy, which is anticancer immunotherapy, of each chromosome in FIG. 4B. In all chromosomes, for the ratio of DMR, the level of pDMR is shown to be higher than that of eDMR.

Referring to FIG. 4C, DMR target genes are involved in many immune-related pathways, and pDMR target genes are also shown to be involved in immune pathways such as human papillomavirus infection.

Referring to FIGS. 4D to 4E, the primer information of the gene and the gene selected by the method described above in FIG. 4A is shown. Referring to FIG. 4D, among differentially methylated promoters, functional DMR appears to have a negative correlation between methylation level and gene expression level. In this connection, when the p value of the Pearson correlation coefficient (PCC), which indicates the correlation between the methylation level and the gene expression level, is less than 0.05 and in the case of a gene with positive methylation change, as indicated by FC<½ (q<0.01) in differential expressed gene analysis (DEG analysis), it has a mean beta FC>0.15 value, and log 2FC (fold change) has a log 2FC>2 value because the gene means expressive power. The FDR (false discovery rate), which indicates the gene error discovery rate, was selected as a reference value with FDR<0.01 and p adjusted value of less than 0.01. Hence, more functionally relevant DMRs, i.e., DMRs in which hypomethylated DMRs simultaneously up-regulate the target gene or hypermethylated DMRs simultaneously down-regulate the target gene, were selected. As a result, pDMRs for CTYIP, TNFSF8 and C11orf21 were selected. The bisulfite pyrosequencing method was used to measure the methylation of the three selected genes, and the sequence information of the primers (SEQ ID NO:1-6, sequentially) used in this connection is shown in FIG. 4E.

The results of Examples 1 to 3 indicate that the therapy response of non-small lung cancer patients can be predicted more accurately based on the DNA methylation measurement of pDMRs. More specifically, the DNA methylation level of pDMRs for at least one gene in the group consisting of CYTIP and TNFSF8 can act as practical indicators for prediction of the response to anti-PD-1 therapy, which is anticancer immunotherapy.

However, the present invention is not limited to the above, and can be used to provide information for the prediction of a therapeutic response to a variety of immunotherapy methods. For example, the present invention can be configured to provide information for prediction of the therapeutic response to at least one therapy selected from the group consisting of anti-CTLA-4 therapy, anti-CD28 therapy, anti-KIR therapy, anti-TCR therapy, anti-LAG-3 therapy, anti-TIM-3 therapy, anti-TIGIT therapy, anti-A2aR therapy, anti-ICOS therapy, anti-OX40 therapy, anti-4-1BB therapy, and anti-GITR therapy.

Furthermore, the present invention can further provide a kit for providing information configured to predict a therapeutic response to anticancer immunotherapy as the kit contains a formulation configured to measure DNA methylation of pDMRs for a biological sample isolated from a subject.

The features of the various embodiments of the present invention can be partially or entirely coupled or combined with each other. As those skilled in the art can fully understand, various technical associations and operations therebetween can be realized. The embodiments may be implemented independently of each other and may be implemented together in a combined relationship.

Although the embodiments of the present invention have been described in more detail with reference to the accompanying drawings, the present invention is not necessarily limited to these embodiments. Various modifications can be made within the scope of the technical idea of the present invention. Therefore, the embodiments disclosed in the present invention are not intended to limit the technical idea of the present invention thereto. The scope of the technical idea of the present invention is not limited to the embodiments. Therefore, it should be understood that the embodiments described above are illustrative in all respects and not limiting. The scope of protection of the present invention should be construed by the claims below. All technical ideas within the scope of the equivalents thereto should be construed as being included in the scope of the present invention.

[National R&D project that supported the present invention] Project Identification Number: 2018R1A5A2025079, Ministry Name: Ministry of Science and Technology Information and Communication, Research Management Organization: Korea Research Foundation, Research Project Name: Leading Research Center Support Project, Research Project Title: Chronic Intractable Disease Systems Medicine Research Center, Contribution Percentage: ¼, Host Institution: Yonsei University Industry-Academic Cooperation Foundation, Research Period: 20180601 to 20220228,

[National R&D project that supported the present invention] Project Identification Number: 2018M3C9A5064709, Ministry Name: Ministry of Science and Technology Information and Communication, Research Management Organization: Korea Research Foundation, Research Project Name: Post-Genome Project, Research Project Title: Development of Network Augmented Analysis Web Service for Genome Big Data Utilization, Contribution Percentage: ¼, Host Institution: Yonsei University Industry-Academic Cooperation Foundation, Research Period: 20180701 to 20211231,

[National R&D project that supported the present invention] Project Identification Number: 2017R1D1A1B03029874, Ministry Name: Ministry of Education, Research Management Organization: Korea Research Foundation, Research Project Name: Subject Basic Research (Ministry of Education) (R&D), Research Project Title Immuno-anticancer drugs using immune markers in peripheral blood of lung cancer patients, Establishment of effective immuno-cancer treatment strategies through identification of treatment predictors, Contribution Percentage: ⅙, Host Institution: Yonsei University Industry-Academic Cooperation Foundation, Research Period: 20190301 to 20200229,

[National R&D project that supported the present invention] Project Identification Number: 2017M3A9E9072669, Ministry Name: Ministry of Science and Technology Information and Communication, Research Management Organization: Korea Research Foundation, Research Project Name: Biomedical Technology Development Project (R&D), Research Project Title: Identification of the mechanism of acquired resistance to anticancer drugs through construction of high-precision preclinical model using patient-derived circulating tumor cells and presentation of treatment strategies, Contribution Percentage: ⅙, Host Institution: Yonsei University Industry-Academic Cooperation Foundation, Research Period: 20190401 to 20200131,

[National R&D project that supported the present invention] Project Identification Number: 2017M3A9E8029717, Ministry Name: Ministry of Science and Technology information and Communication, Research Management Organization: Korea Research Foundation, Research Project Name: Biomedical Technology Development Project (R&D), Research Project Title: Precise prediction of therapeutic effects and side effects of advanced lung cancer using genes and immune biomarkers, Contribution Percentage: ⅙, Host institution: Yonsei University Industry-Academic Cooperation Foundation, Research Period: 20190101 to 20191231.

1. A method for providing information about a therapeutic response to anticancer immunotherapy, the method comprising:
measuring DNA methylation of pDMRs (promotor associated differentially methylated regions) in a biological sample isolated from a subject; and evaluating the therapeutic response to the anticancer immunotherapy for the subject on the basis of the measured DNA methylation of pDMRs.

2. The method according to claim 1, wherein:
the subject is a subject suspected of non-small lung cancer; and
the biological sample includes at least one selected from the group consisting of tumor tissue, blood, serum, and plasma, and is isolated from the subject before the anticancer immunotherapy is performed.

3. The method according to claim 1, wherein measuring DNA methylation of pDMRs includes measuring presence or absence of the DNA methylation, or methylation level of pDMRs.

4. The method according to claim 3, wherein evaluating the therapeutic response to anticancer immunotherapy includes:
determining the subject as positive, when the measured DNA methylation level of pDMRs for at least one gene in the group consisting of CYTIP and TNFSF8 is less than a predetermined level, and determining the subject as negative when the measured DNA methylation level is greater than or equal to a predetermined level; and
evaluating the therapeutic response to the anticancer immunotherapy, based on whether the DNA methylation level is positive or negative.

5. The method according to claim 4, wherein:
the gene includes the at least one CYTIP gene; and
evaluating the therapeutic response includes: evaluating the subject as positive for the therapeutic response to the anticancer immunotherapy, when the DNA methylation level of pDMRs for the CYTIP gene in the biological sample is less than 50%.

6. The method according to claim 4, wherein:
the gene includes the at least one TNFSF8 gene; and
evaluating the therapeutic response includes: evaluating the subject as positive for the therapeutic response to the anticancer immunotherapy, when the DNA methylation level of pDMRs for the TNFSF8 gene in the biological sample is less than 60%.

7. The method according to claim 4, wherein:
the gene includes a combination of the CYTIP and TNFSF8 genes; and
evaluating the therapeutic response includes: evaluating the subject as positive for the therapeutic response to the anticancer immunotherapy, when the DNA methylation level of pDMRs for a combination of the CYTIP and TNFSF8 genes in the biological sample is less than a predetermined level.

8. The method according to claim 1, wherein the anticancer immunotherapy includes at least one therapy selected from the group consisting of anti-CTLA-4 therapy, anti-PD-1 therapy, anti-CD28 therapy, anti-KIR therapy, anti-TCR therapy, anti-LAG-3 therapy, anti-TIM-3 therapy, anti-TIGIT therapy, anti-A2aR therapy, anti-ICOS therapy, anti-OX40 therapy, anti-4-1BB therapy, and anti-GITR therapy.

9. A kit for providing information about a therapeutic response to anticancer immunotherapy, the kit comprising formulations configured to measure DNA methylation of pDMRs (promotor associated differentially methylated regions) in a biological sample isolated from a subject.

10. The kit according to claim 9, wherein:
the subject is a subject suspected of non-small lung cancer; and
the biological sample includes at least one selected from the group consisting of tumor tissue, blood, serum, and plasma, and is isolated from the subject before the anticancer immunotherapy is performed.

11. The kit according to claim 9, wherein the measurement of DNA methylation of pDMRs includes measuring presence or absence of the DNA methylation, or methylation level of pDMRs.

12. The kit according to claim 9, wherein:
the formulation includes formulations configured to measure the DNA methylation of pDMRs for at least one gene from the group consisting of CYTIP and TNFSF8 in the biological sample isolated from the subject; and
the kit is configured to present positive or negative therapeutic response to anticancer immunotherapy for the subject based on the measured DNA methylation of pDMRs.

13. The kit according to claim 12, wherein:
the formulation includes at least one formulation configured to measure the DNA methylation level of pDMRs for the CYTIP gene; and
the kit is configured to present the subject as positive for the therapeutic response to the anticancer immunotherapy, when the DNA methylation level of pDMRs for the CYTIP gene in the biological sample is less than 50% and greater than or equal to the kit is further configured to present the subject as negative for the therapeutic response to the anticancer immunotherapy, when the DNA methylation level of pDMRs for the CYTIP gene is greater than or equal to 50%.

14. The kit according to claim 12, wherein:
the formulation includes at least one formulation configured to measure the DNA methylation level of pDMRs for the TNFSF8 gene; and
the kit is configured to present the subject as positive for the therapeutic response to the anticancer immunotherapy, when the DNA methylation level of pDMRs for the TNFSF8 gene in the biological sample is less than 60% and greater than or equal to the kit is further configured to present the subject as negative for the therapeutic response to the anticancer immunotherapy, when the DNA methylation level of pDMRs for the TNFSF8 gene is greater than or equal to 60%.

15. The kit according to claim 12, wherein:
the formulation includes formulations configured to measure the DNA methylation level of pDMRs for a combination of the CYTIP and TNFSF8 genes; and
the kit is configured to present the subject as positive for the therapeutic response to the anticancer immunotherapy, when the DNA methylation level of pDMRs for a combination of the CYTIP and TNFSF8 genes in the biological sample is less than a predetermined level and the kit is further configured to present the subject as negative for the therapeutic response to the anticancer immunotherapy, when the DNA methylation level of pDMRs for a combination of the CYTIP and TNFSF8 genes is greater than or equal to a predetermined level.

16. The kit according to claim 9, wherein the anticancer immunotherapy includes at least one therapy selected from the group consisting of anti-CTLA-4 therapy, anti-PD-1 therapy, anti-CD28 therapy, anti-KIR therapy, anti-TCR therapy, anti-LAG-3 therapy, anti-TIM-3 therapy, anti-TIGIT therapy, anti-A2aR therapy, anti-ICOS therapy, anti-OX40 therapy, anti-4-1BB therapy, and anti-GITR therapy.


SEQUENCE LISTING (6 sequences)

SEQ ID NO: 1 - DNA, 22 bp, Homo sapiens
ggtgagtgtggagtaagagtta

SEQ ID NO: 2 - DNA, 23 bp, Homo sapiens
cccatcctacaaaaccactcaac

SEQ ID NO: 3 - DNA, 20 bp, Homo sapiens
agtgtggagtaagagttata

SEQ ID NO: 4 - DNA, 26 bp, Homo sapiens
gttagagtggttgtggttaaatagaa

SEQ ID NO: 5 - DNA, 27 bp, Homo sapiens
caaacactcaaccaaataacccctcct

SEQ ID NO: 6 - DNA, 16 bp, Homo sapiens
gtttttaggtggttgg